(19) United States
(12) Patent Application Publication
(10) Pub. No.: US 2017/0043297 A1
(43) Pub. Date: Feb. 16, 2017

(54) ASYMMETRIC POLY(PHENYLENE ETHER) CO-POLYMER MEMBRANE, SEPARATION MODULE THEREOF; AND METHODS OF MAKING

(71) Applicant: SABIC GLOBAL TECHNOLOGIES B.V., Bergen op Zoom (NL)

(72) Inventors: Albin Peter Berzinis, Delmar, NY (US); Rachel Elizabeth Halbfinger, Glenville, NY (US); Matias Bikel, Bergen op Zoom (NL); Pooja Bajaj, Schenectady, NY (US)

(21) Appl. No.: 15/303,061

(22) PCT Filed: May 1, 2015
(86) PCT No.: PCT/US2015/028842

§ 371 Date: Oct. 10, 2016

Related U.S. Application Data
(60) Provisional application No. 61/987,168, filed on May 1, 2014.

(51) Int. Cl. B01D 61/14; B01D 61/24; B01D 61/28; B01D 61/36; B01D 63/10; B01D 67/00; B01D 69/06; B01D 69/08; B01D 69/12; B01D 63/02; B01D 71/52; B01D 69/02; B01D 71/76; C07K 1/34; C08B 37/00; A61M 1/16; B01D 61/18

(52) U.S. Cl. CPC B01D 61/145; B01D 61/18; B01D 61/243; B01D 61/28; B01D 61/364; B01D 61/366; B01D 63/10; B01D 67/0016; B01D 69/06; B01D 69/08; B01D 69/12; B01D 63/02; B01D 71/52; B01D 69/02; B01D 71/76; C07K 1/34; C08B 37/0003; A61M 1/1623; A61M 1/1698; B01D 2325/022; B01D 2325/34

(57) ABSTRACT

A porous membrane made from a poly(phenylene ether) copolymer has at least one of: a molecular weight cut off of less than 40 kilodaltons or a surface pore size of 0.001 to 0.1 micrometers. The porous membrane is made by dissolving the poly(phenylene ether) copolymer in a water-miscible polar aprotic solvent to form a porous membrane-forming composition; and phase-inverting the porous asymmetric membrane forming-composition in a first non-solvent composition to form the porous mem-brane. The porous membrane can be in the form of a sheet or a hollow fiber, and can be fabricated into separation modules.

### BACKGROUND OF THE INVENTION

Ultrafiltration is a membrane separation process whereby a feed stock containing a solute, which has molecular or colloidal dimensions which are significantly greater than the molecular dimensions of its solvent, is depleted of the solute by being contacted with the membrane at such pressure that the solvent permeates the membrane and the solute is retained. This results in a permeate fraction which is solute depleted and a retentate fraction which is solute enriched. In ultrafiltration, and similarly nanofiltration and microfiltration, pressure in excess of the osmotic pressure can be used to force the solvent through the membrane. Reverse osmosis for drinking water production, the production of milk protein concentrate for cheese production, and enzyme recovery are examples.

A commercially viable separation membrane combines high selectivity, high permeation flux or throughput, and a long service life. Permeation flux is a measure of volumetric permeate flow through a membrane. The higher the permeation flux, the smaller the membrane area required to treat a given volume of process fluid. Separation factor is a measure of membrane selectivity. Separation factor is the ratio of the flux of the permeate across the membrane to the flux of the process stream. Since selectivity can be inversely proportional to flux, it is desirable to increase the selectivity without adversely affecting flux. It is also desirable to have separation membranes with long service lives under harsh conditions, for example high temperatures and exposure to corrosive reagents, so that replacement costs are minimized. A large number of materials have been investigated for use in separation membranes for reverse osmosis.

Poly(phenylene ether)s are a class of plastics having excellent water resistance, thermal resistance, and dimensional stability. They retain their mechanical strength in hot, and/or wet environments. Therefore they can be used for the fabrication of porous membranes useful in various separation processes. For example, poly(phenylene ether)s can be used in processes that require repeated cleaning with hot water or steam sterilization. Nonetheless, there remains a need for a membrane having improved filtration properties, including materials that will improve selectivity without adversely affecting permeation flux.

### BRIEF DESCRIPTION OF THE INVENTION

A porous membrane comprises, consists essentially of, or consists of a poly(phenylene ether) copolymer, wherein the porous membrane has at least one of a molecular weight cut off of less than 40 kilodaltons and a surface pore size of 0.001 to 0.1 micrometers. A method of making the porous membrane comprises: dissolving the poly(phenylene ether) copolymer in a water-miscible polar aprotic solvent to form a porous membrane-forming composition; and phase-inverting the porous asymmetric membrane forming-composition in a first non-solvent composition to form the porous membrane. A porous membrane is made by the method, and the porous membrane can be fabricated into a separation module.

A method of making a hollow fiber by coextrusion through a spinneret comprising an annulus and a bore, wherein the method comprises coextruding: a membrane-forming composition comprising a poly(phenylene ether) copolymer, dissolved in a water-miscible polar aprotic solvent through the annulus, and a first non-solvent composition comprising water, a water-miscible polar aprotic solvent, or a combination comprising at least one of the foregoing, through the bore, into a second non-solvent composition comprising water, a water-miscible polar aprotic solvent, or a combination comprising at least one of the foregoing, to form the hollow fiber. A hollow fiber is made by the method, and can be fabricated into a separation module.

### BRIEF DESCRIPTION OF THE DRAWINGS

Referring now to the drawings: FIG. 1 depicts SEM images of the hollow fiber membranes of Comparative Example 3 and Example 18.

FIG. 2 depicts a diagram of a laboratory scale, dry-wet immersion precipitation hollow fiber spinning apparatus.

FIG. 3 shows an embodiment of a hollow fiber separation module.

FIG. 4 shows another embodiment of a hollow fiber separation module.

FIG. 5 shows an embodiment of a spiral wound separation module.

FIG. 6 shows an embodiment of a disk separation module.

FIG. 7 shows an embodiment of a plate and frame separation module.

FIG. 8 shows an embodiment of a system for pretreatment of water.

FIG. 9 shows an embodiment of a separation module for wastewater treatment.

FIG. 10 shows an embodiment of a system for wastewater treatment.

FIG. 11 shows an embodiment of a module for membrane distillation.

### DETAILED DESCRIPTION OF THE INVENTION

The inventors hereof have discovered that a specific class of copolymers having two or more different types of poly(phenylene ether) repeat units is particularly useful in the manufacture of porous membranes for ultrafiltration. The poly(phenylene ether) copolymer is hydrophobic, and can be fabricated into both flat membranes and hollow fiber membranes. Thus, the porous membrane comprises, consists essentially of, or consists of a poly(phenylene ether) copolymer, wherein the porous membrane has at least one of a molecular weight cut off of less than 40 kilodaltons and a surface pore size of 0.001 to 0.1 micrometers. The hydrophobic polymer can be a poly(phenylene ether) copolymer having an intrinsic viscosity greater than or equal to 0.7, 0.8, 0.9, 1.0, or 1.1 deciliters per gram (dL/g), and less than or equal to 1.5, 1.4, or 1.3 dL/g, when measured in chloroform at 25° C. In some embodiments, the intrinsic viscosity is 1.1 to 1.3 dL/g. In some embodiments, the poly(phenylene ether) copolymer has a weight average molecular weight of 100,000 to 500,000 daltons (Da), as measured by gel permeation chromatography against polystyrene standards. Within this range, the weight average molecular weight can be greater than or equal to 150,000 or 200,000 Da and less than or equal to 400,000, 350,000, or 300,000 Da. In some embodiments, the weight average molecular weight is 100,000 to 400,000 Da, specifically 200,000 to 300,000 Da. The poly(phenylene ether) copolymer can have a polydispersity (ratio of weight average molecular weight to number average molecular weight of 3 to 12. Within this range, the polydispersity can be greater than or equal to 4 or 5 and less than or equal to 10, 9, or 8.

in some embodiments, the poly(phenyiene ether) copolymer has a solubility of 50 to 400 grams per kilogram in N-methyl-2-pyrrolidone at 25° C., based on the combined weight of the poly(phenylene ether) copolymer and NMP. Within this range, the solubility can be greater than or equal to 100, 120, 140, or 160 grams per kilogram (g/kg), and less than or equal to 300, 250, 200, or 180 g/kg at 25° C. Advantageously, a copolymer having an intrinsic viscosity of 0.7 to 1.5 dfig, specifically 1.1 to 1.3 dlLig, and a solubility of 50 to 400 g/kg provides membrane-forming compositions conducive to the formation of suitable porous membranes in the absence of polymer additives such as :hydrophilic polymers, for example, poly(N-vinylpyrrolidone), which can serve as a viscosity modifier.

The porous membrane has many advantageous properties. The poly(phenylene ether) copolymers have hydrophobic surfaces, as measured, for example, by water contact angle. Because of the hydrophobic surface, the porous membranes can be used for purification of a variety of aqueous and non-aqueous streams and gaseous streams, and are resistant to fouling. Advantageously, the copolymer has a. desirable pore size distribution, membrane selectivity, and permeation flux. The poly(phenyiene ether) copolymer further resists extraction by water. Advantageously, this results in reduced loss of poly(phenylene ether) copolymer upon contact with process streams in end-use applications, and especially during cleaning.

Polymer additives, such as hydrophilic polymers, have been added to membrane-forming compositions to impart a viscosity to the membrane-forming compositions that is conducive to the formation of a porous membrane useful for purification of aqueous streams. However, hydrophilic polymers, when present in the porous asymmetric membrane, are prone to extraction in the phase inversion and washing steps of membrane fabrication. Moreover the hydrophilic polymer can be leached out of the membrane in the end-use application—membrane treatment of aqueous streams. For example, polyethersulfone can be blended with poly(N-vinylpyrrolidone), and the two polymers can be co-precipitated from solution to form a membrane. Excess poly(N-vinylpyrrolidone) must be washed off of the membrane with water, which results in a waste of valuable material, and which produces an aqueous waste comprising the excess poly(N-vinylpyrrolidone).

Advantageously, the porous membranes are useful for purification of aqueous or non-aqueous streams, and are produced in the absence of hydrophilic or amphiphilic polymers, or any other viscosity modifier. Thus, in some embodiments, hydrophilic and amphiphilic polymers are absent from the membrane-forming composition and the first non-solvent composition. An amphiphilic polymer is defined herein as a polymer that has both hydrophilic (water-loving, polar) and hydrophobic (water-hating, non-polar) properties. For example, the amphiphilic polymer can be a block copolymer comprising a hydrophobic block and a hydrophilic block or graft. The hydrophilic and amphiphilic polymers absent from the membrane-forming composition and the first non-solvent composition can comprise, for example, poly(vinyl pyrrolidone), poiy(oxazoline), poly(ethylene glycol), poly(propylene glycol), a poly(ethylene glycol) monoether or monoester, a polypropylene glycol) monoether or monoester, a block copolymer of poly(ethylene oxide) and poly(propylene oxide), polystyrene-graft-poly(ethylene glycol), polystyrene-graft-poly(propylene glycol), polysorbate, cellulose acetate, or a combination comprising at least one of the foregoing.

The water-miscible polar aprotic solvent is one that is polar, but does not have any ionizable hydrogen atoms at a pH of 1 to 14. The water-miscible polar aprotic solvent can be, for example, N-methyl-2-pyrrolidone (NMP), N,N-dimethylformamide (DMF), N,N-dimethylacetamide (DMAC), N-ethyl-2-pyrrolidone, dimethyl sulfoxide (DMSO), dimethyl sulfone, sulfolane, butyrolactone; and combinations comprising at least one of the foregoing. In some embodiments, the water-miscible polar aprotic solvent comprises N-methyl-2-pyrrolidone.

The first non-solvent composition serves as a coagulation, or phase inversion, bath for the porous membrane-forming composition. The porous membrane is formed by contacting the membrane-forming composition with the first non-solvent composition. The poly(phenylene ether) copolymer, which is near its gel point in the membrane-forming composition, coagulates, or precipitates as a film or hollow fiber depending upon the specific method used. The second non-solvent composition serves to rinse residual water-miscible solvent, if present, from the membrane. The first and second non-solvent compositions can be the same or different, and can comprise water, or a mixture of water and a water-miscible polar aprotic solvent. In sonic embodiments the first and second non-solvents are independently selected from water, and a mixture of water and NMP. In sonic embodiments, the first and second non-solvent compositions are both water. The water can be deionized.

In some embodiments, the first non-solvent composition comprises 10 to 100 wt. % water and 0 to 90 wt. % NMP, based on the total weight of the first non-solvent composition. Within this range, the first non-solvent composition can comprise 10 to 90 weight percent, specifically 10 to 80 wt. % , water and 10 to 90 wt. % , specifically 20 to 90 wt. % , NMP. In some embodiments, the first non-solvent composition comprises about 70 wt. % water and about 30 wt. % NMP.

Any of several techniques for the phase inversion step of porous membrane forrnation can be used, For example, the phase inversion step can be a dry-phase separation method in which the dissolved copolymer is precipitated by evaporation of a sufficient amount of solvent mixture to form the membrane The phase inversion step can also be a wet-phase separation method in which the dissolved copolymer is precipitated by immersion in the first non-solvent to form the membrane, The phase inversion step can be a dry-wet phase separation method, which is a combination of the dry-phase and the wet-phase methods. The phase inversion step can be a thermally-induced separation method in which the dissolved copolymer is precipitated or coagulated by controlled cooling to form the membrane. The membrane, once formed, can be subjected to membrane conditioning or pretreatment, prior to its end-use. The conditioning or pretreatment can be thermal annealing to relieve stresses or pre-equilibration in the expected feed stream.

The method is applicable to hollow fiber spinning. Thus in some embodiments, a method of making a hollow fiber by coextrusion through a spinneret comprising an annulus and a bore, comprises coextruding: a membrane-forming composition comprising a poly(phenylene ether) copolymer, dissolved in a water-miscible polar aprotic solvent through the annulus, and a first non-solvent composition comprising water, a water-miscible polar aprotic solvent, or a combination comprising at least one of the foregoing, through the bore, into a second non-solvent composition comprising water, a water-miscible polar aprotic solvent, or a combination comprising at least one of the foregoing, to form the hollow fiber. In some embodiments of the method of making a hollow fiber, hydrophilic and amphiphilic polymers are absent from the membrane-forming composition and the first non-solvent composition. In some embodiments the hollow fiber is made by the method, in which hydrophilic and amphiphilic polymers are absent from the membrane-forming composition and the first and second non-solvent compositions.

The porous asymmetric membrane made by the method can have a mean surface pore size distribution on the selective layer of greater than or equal to 1, 5. 10 nanometers (nm) and less than or equal to 100, 50, or 20 nm ±1, 2, 5, or 10 nm. The porous asymmetric membrane made by the method can also have a surface pore density of greater than or equal to 100, 200, or 400 pores per μm² and less than or equal to 4,000, 2,400, or 1,200 pores per μm^{2.}

The configuration of the porous asymmetric membrane made by the method can be sheet, disc, spiral wound, plate and frame, hollow fiber, capillary, or tubular. Outside-in and inside-out separations are applicable to hollow fiber membranes, capillary membranes, and tubular membranes, each having an inner and outer surface in contact with the feed and retentate or the permeate.

The porous asymmetric membrane made by the method can be a porous hollow fiber. The wall thickness of the hollow fiber can be 20 to 100 micrometers (μm). Within this range, the wall thickness can be greater than 30 and less than or equal to 80, 60, 40 or 35 μm. In another embodiment the fiber diameter can be 50 to 3000 a μm, specifically 100 to 2000 μm. The membrane can comprise a substantially non-porous surface layer, and the non-porous surface layer can be on the inside surface of the hollow fiber. A separation module can comprise bundles of porous hollow fibers. In some embodiments, the fiber bundle comprises 10 to 10,000 porous hollow fibers, The hollow fibers can be bundled longitudinally, potted in a curable resin on both ends, and encased in a pressure vessel to form the hollow fiber module. Hollow fiber modules can be mounted vertically or horizontally.

The porous asymmetric membranes can be fabricated into separation modules designed for purification of various aqueous, non-aqueous (e.g., hydrocarbon), or gaseous streams. Thus in some embodiments, a separation module comprises the porous asymmetric membrane comprising, consisting essentially of, or consisting of: a hydrophobic polymer comprising, consisting essentially of, or consisting of a poly(phenylene ether) or poly(phenylene ether) copolymer and a polymer additive. The separation module can be designed for dead-end separation, cross-flow separation, inside-out separation, or outside-in separation.

Depending upon porous asymmetric membrane surface pore size distribution and pore density, and the end-use, the separation module fabricated from the porous asymmetric membrane made by the method can be a media filtration module, a microfiltration module, an ultrafiltration module, a nanofiltration module, or a reverse osmosis module. The separation module fabricated from the porous asymmetric membrane made by the method can also be a membrane contactors module, a pervaporation module, a dialysis module, an osmosis module, an electrodialysis module, a membrane electrolysis module, an electrophoresis module, or a membrane distillation module. For media filtration, the surface pore size can be about 100 to about 1,000 μm. For microfiltration, the surface pore size can be about 0.03 to about 10 μm. For ultrafiltration, the surface pore size can be about 0.002 to 0.1 μm. For nanofiltration, the surface pore size can be about 0.001 to about 0.002 μm. The porous asymmetric membranes described herein are surprisingly well suited for ultrafiltration and nanofiltration. In some embodiments, the porous asymmetric membrane has a surface pore size of 0.001 to 0.05 μm, specifically 0.005 to 0.01 μm.

The molecular weight cut off (MWCO) of a membrane is the lowest molecular weight solute in which 90 weight percent (wt. %) or greater of the solute is retained by the membrane. The porous asymmetric membranes made by the method can have a MWCO of 500 to 40,000 Da, specifically 1,000 to 10,000 Da, more specifically 2,000 to 8,000 Da, or still more specifically 3,000 to 7,000 Da. Furthermore, any of the foregoing MWCO ranges can be present in combination with a desirable permeate flux, such as clean water permeate flux (CWF). For example, the permeate flux can be 1 to 200, specifically 2 to 100, more specifically 4 to 50 L(h·m²·bar), wherein L is liters and m² is square meters. The porous asymmetric membranes made by the method can also provide a CWF of about 10 to about 80 L/(h·m²·bar), about 20 to about 80 L/(h·m²·bar), or about 40 to about 60 L/(h·rn²·bar). Flux across the membrane is driven by the osmotic or absolute pressure differential across the membrane, referred to herein as the trans-membrane pressure (TMP). The trans-membrane pressure can be 1 to 500 kilopascals (kPa), specifically 2 to 400 kPa, and more specifically 4 to 300 kPa.

The porous asymmetric membranes disclosed herein are useful for treatment of aqueous streams. Depending upon pore size and porous asymmetric membrane configuration, the membranes can be used to remove suspended matter, particulate matter, sands, silt, clays, cysts, algae, microorganisms, bacteria, viruses, colloidal matter, synthetic and naturally occurring macromolecules, dissolved organic compounds, salts, or a combination comprising at least one of the foregoing. Thus, the porous asymmetric membranes disclosed herein can be used in wastewater treatment, water purification, food processing, the dairy industry, biotechnology, pharmaceuticals, and healthcare.

The pharmaceutical or biotechnological processes or food processing applications can include, for instance, the removal of salts and/or low molecular weight byproducts from solutions (product streams) by way of dialysis or increasing the concentration of a product having a molecular weight above the cut-off of the membrane in a solution by way of ultrafiltration, such solutions including human blood, animal blood, lymph fluids, or microbial or cellular suspensions (e.g. bacterial, plant cells, animal blood or lymph fluids, or microbial or cellular suspensions). Specific applications include the concentration and purification of peptides in blood plasma; hemofiltration; hemodialysis; hemodiafiltration; renal dialysis; and enzyme recovery. Food processing can involve solutions such as meat products and by-products, plant extracts, suspensions of algae or fungi, vegetable food and beverages containing particles such as pulp, milk processing, cheese processing, and sugar clarification. Specific examples include downstream processing of fermentation broths; concentration of protein in milk, whole egg or egg white with simultaneous removal of salts and sugars; and concentration of gelling agents and thickeners like agar, carrageen, pectins, or gelatin. Thus the module is useful for many different fluid separation applications in a variety of fields in the medical, pharmaceutical, industrial, and food industries.

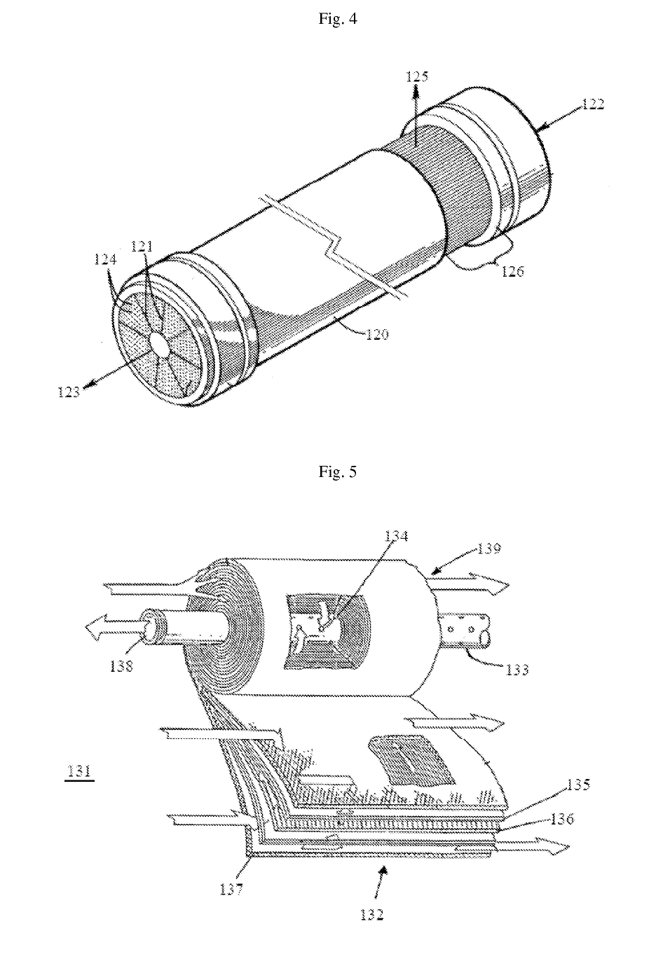

Disclosed in FIG. 4 is an embodiment of a separation module 110 comprising one or more bundles of hollow fibers of the asymmetric membrane. Each fiber bundle may be contained within an enclosure 111 that is substantially impermeable to the fluids to be separated to prevent fluid from passing between adjacent fiber bundles, The hollow fibers may be embedded in and communicate through an encasement 116 at either end of the module. The encasement may comprise a thermoset, such as epoxy, polyester, melamine, polysiloxane, or a polyurethane; or may comprise a thermoplastic, such as polyethylene, polypropylene, poly(ethylene terephthalate), or poly(1,4-butylene terephthalate), for example, The feed stream 112 enters the bore of the fibers at one end of the module and the retentate stream 113 leaves at the opposite end. The encasement may be disposed at ends of the bundles for attaching and sealing to the bundles. The permeate 114 can be recovered from holes 115 disposed in a side of the enclosure, alternatively the permeate may be recovered from holes in the encasement.

The fiber bundles need not be cylindrical. For example, shown in FIG. 5 is an embodiment of a separation module 120 in which the bundles of fibers are separated by an impermeable barrier 121. In the separation module 120, the feed stream 122 enters the bores of the hollow fibers at one end of the bundles 124 and the retentate stream 123 exits at the opposite end. The permeate fluid 125 can exit the module through an opening 126 in a side of the module.

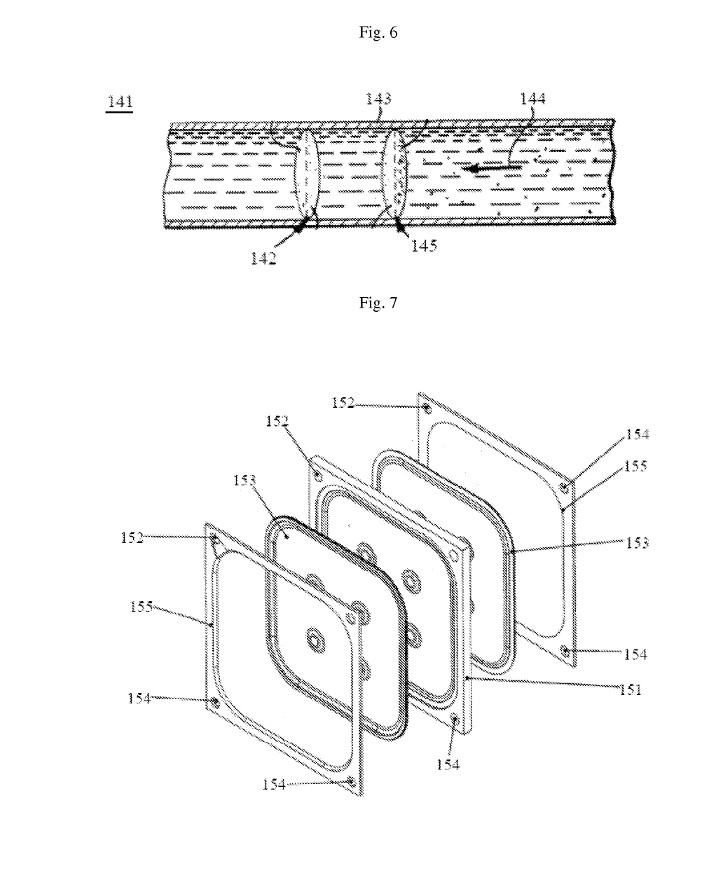

The separation module may have a spiral wound design, as shown in FIG. 6. A spiral wound separation module 131 may comprise a sheet of the asymmetric membrane 132 wound onto a hollow core member 133 having perforations 134, Alternatively, the hollow core member 133 may comprise a porous material. Additional layers, such as reinforcing layer 135, inner spacer 136, and outer spacer 137 are also provided. The permeated fluid passes through the perforations 134 in the hollow core member 133 and can be removed through the output 138 of the hollow core member 133. Retentate fluid passes through the outer spacer 137 and exits through the residual output 139.

The separation module may have a disk design, as shown in FIG. 7, A disk separation module 141 may comprise a filter 142 comprising the asymmetric membrane disposed within a tube 143. The tube may comprise any suitable material, such as a material that is impermeable to the fluid. A support (not shown) may be optionally present. The fluid 144 may contact the disk at a selected pressure sufficient to cause the permeate to pass through the disk. In another embodiment, a plurality of disks may be used, for example to provide a prefilter 145. The prefilter 145 may be the same as or different than the filter 142. For example, the prefilter 145 may have larger pores than the filter 142, or the prefilter 145 may further comprise a functionalized surface, e.g., a surface having a catalyst disposed thereon. In another embodiment the prefilter 145 comprises the asymmetric membrane and the filter 142 comprises a different material.

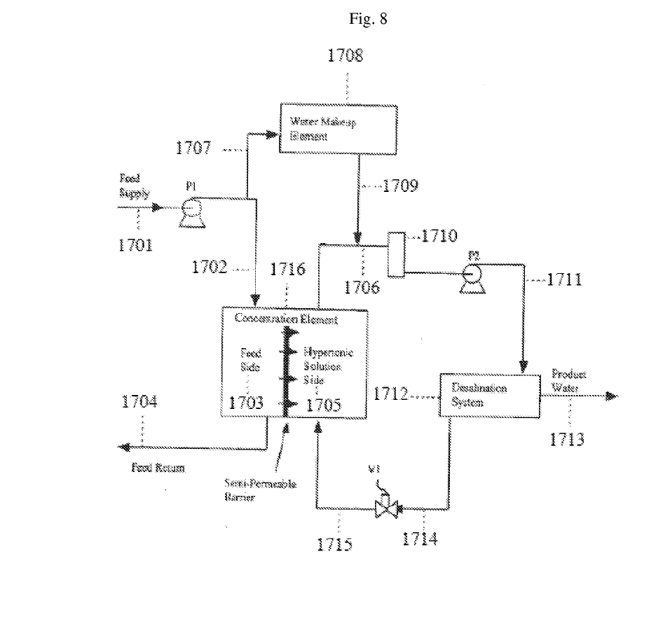

The separation module may have a plate and frame design, as shown in the expanded view of FIG. 8. A filter plate of the separation module may comprise a base body 151, the asymmetric membrane 153, and a frame 155, wherein the frame 155 comprises an inlet 152 and an outlet 154. The asymmetric membrane is mounted on one or both sides of the base body and is held in place by a frame mounted in face to face contact with the asymmetric membrane to form the filter plate. The filter can have any suitable shape, and can be square, round, rectangular, or polygonal. The inlet and outlet allow entry of the input stream and exit of the permeate stream. An advantage of the plate and frame design is that the filter media used in making the filter plate assembly can be replaced when desired. The frame 155 and base body may comprise any suitable material, such as a metal, such as steel, or aluminum, or a polymer such as polypropylene or polyethylene. The frame 155 may be fabricated by a molding or a casting process and then machined to the desired size. Due to the solid nature of the frame 155, it can hold the asymmetric membrane 153 to the base body 151 tightly and provide a desirable sealing effect.

The production of purified water, e.g., drinking water, is also disclosed. Reverse osmosis membranes are designed to remove dissolved salts from water. Water passes readily through the reverse osmosis membrane, whereas dissolved salt is retained. Under natural conditions of osmosis, water will diffuse through a semipermeable membrane toward a region of higher salt concentration in order to equalize solution strength on both sides of the membrane. In order to overcome and reverse this osmotic tendency, pressure is applied to feedwater to force water to permeate from a region of higher salt concentration to lower salt concentration, thereby producing a purified stream.

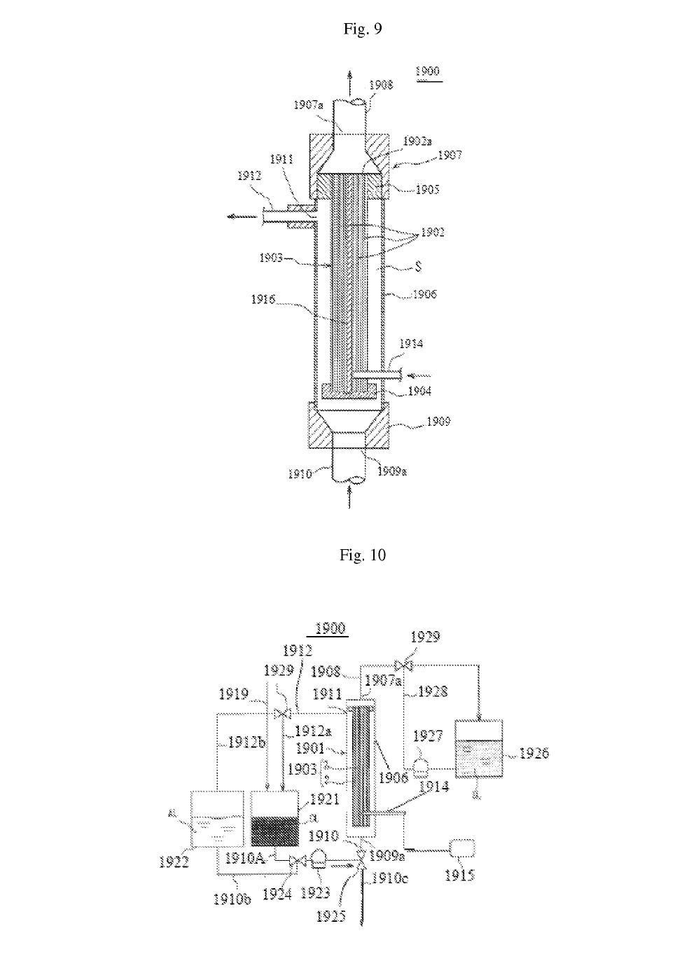

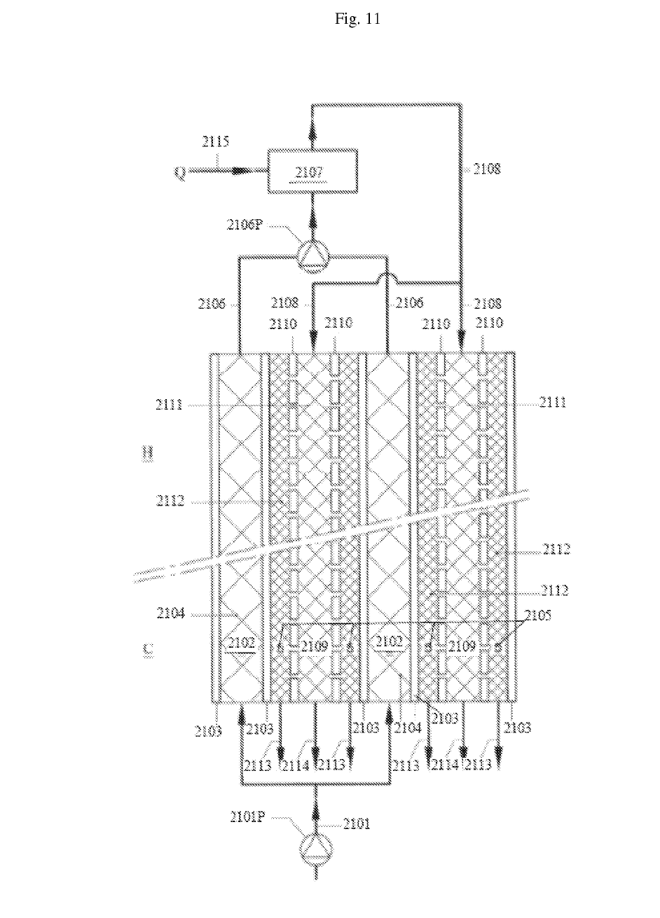

The membrane may have particular application pretreatment of water in a desalination system, an embodiment of which is shown in FIG. 10. The pretreatment may remove or treat any solutes that may that may foul or scale the desalination element. A hypertonic solution can comprise a concentrated feed and its solute composition is the same as that in the feed. However, in a hypertonic solution, contamination components have been concentrated to a level higher than the feed. Additionally, in the hypertonic solution, any solutes that may foul or scale the desalination element can be removed or in some cases addressed by the introduction of anti-scale agents. The membrane separates the feed 173 and the hypertonic solution 175. In such an embodiment, water flows from the feed into the hypertonic solution across the membrane according to water concentration gradients from the feed to the hypertonic solution. Thus, the feed water can be concentrated and the hypertonic solution can be diluted in a concentration module 1716 which comprises the membrane. The hypertonic solution can then be re-concentrated in a desalination system by distillation, electrodialysis or otherwise and then recycled back into a concentration module comprising the membrane. As shown in FIG. 10, feed 171 enters the system through pump P1 and exits by a discharge 174. In the concentration element, the feed 172 can be passed across one side of the separation membrane on the feed side of the concentration module 173. On the permeate side of the membrane is a hypertonic solution, The hypertonic solution can comprise feed water that has been concentrated to a level higher than the feed but lower than its solubility threshold. In the concentration element, water diffuses along concentration gradients from the higher liquid content feed 173 through the membrane and into the lower liquid content hypertonic solution 175. The feed 172 can therefore be concentrated and the hypertonic solution 5 can be diluted in the concentration module 1716. In the desalination system water can be removed from the hypertonic solution. This water becomes the product 1713 of the overall process. The hypertonic solution can be re-concentrated as a result of the removal of the product water. This reconstituted hypertonic solution 1714 can be then passed through valve V1 and returned back to the hypertonic solution side of the concentration module 1715 and the process can be repeated. Lost solute can be made-up by diverting a constant flow of feed 177 back into the hypertonic solution 179. After mixing the feed makeup 179 with the hypertonic solution 176 it can be passed into a permeate holding tank 1710. From tank 1710 the fluid can then be pumped by P2 via 1711 into the desalination system 1712.

Similarly, the module may be used to remove contaminants, including biological contaminants such as bacteria or protozoa, or organic contaminants, such as organic compounds such as polychlorinated biphenyls (PCBs), to produce a purified product stream.

The porous asymmetric membrane is also useful for wastewater treatment. An embodiment of a separation module for treatment of oil-containing wastewater is shown in FIG. 12, and a system for wastewater treatment comprising the separation module for oil-containing wastewater treatment is shown in FIG. 13. The separation membrane module 191 includes an assembly 193 in which a plurality of hollow fiber membranes 192 are gathered together, the lower end of the assembly 193 is sealed with a sealing member 194, and a lower open end of each hollow fiber membrane 192 is closed. The upper end of the assembly 193 is fixed with a fixing member 195 with an upper open end 192a of each hollow fiber membrane 192 being in an open state. The assembly 193 is housed in an external cylinder 196. An upper cap 197 is attached by bonding to the upper end of the external cylinder 196. An outlet port 197a is provided such that the inside of the upper cap 197 communicates with the hollow portion of each hollow fiber membrane 192, and the outlet port 197a is connected to an outlet pipe 198 for taking out treated liquid. A lower cap 199 is fixed by bonding to the lower end of the external cylinder 196. An inlet port 199a for liquid to be treated, i.e., oil-containing wastewater, is provided on the lower cap 199, and the inlet port 199a is connected to an inlet pipe 1910 which introduces the liquid to be treated. A space S is secured between the sealing member 4 at the lower end of the assembly 193 and the inner wall of the external cylinder 196 so that oil-containing wastewater introduced from the inlet port 199a can promptly flow into the assembly 3 inside the external cylinder 196. Furthermore, a discharge port 1911 for non-filtered, untreated liquid is provided at the peripheral wall in the vicinity of the upper end of the external cylinder 196, and the discharge port 1911 communicates with a circulation pipe 1912. Furthermore, the upper fixing member 195 and the lower sealing member 194 are joined by a reinforcing bar 1916 at the center. The reinforcing bar 1916 prevents the non-rigid hollow fiber membranes 192 from being lifted by the force of stream of oil-containing wastewater from the lower part and is provided to secure verticality. Regarding the system for wastewater treatment as shown in FIG. 13, system 1920 includes an oil-containing wastewater storage tank 1921 into which oil-containing wastewater OL is continuously poured from a pipe 1919, a cleaning liquid storage tank 1922 which stores a cleaning liquid composed of an alkaline aqueous solution (hereinafter referred to as an “alkaline cleaning liquid”) AL, the inlet pipe 1910 through which each of the oil-containing wastewater storage tank 1921 and the cleaning liquid storage tank 1922 communicates with an inlet portion 199a for liquid to be treated of the separation membrane module, 191 and which is inserted with a pump 1923 and a switching valve 1924, and the circulation pipe 1912 through which each of the oil-containing wastewater storage tank 1921 and the cleaning liquid storage tank 1922 communicates with the discharge port 1911 for non-filtered, untreated liquid of the separation membrane module 1. The inlet pipe 1910 is provided with the switching valve 1924 on the upstream side of the pump 1923, and the pipe is branched by the switching valve 1924. A branch pipe 19A is connected to the oil-containing wastewater storage tank 1921, and a branch pipe 1910B is connected to the cleaning liquid storage tank 1921 Furthermore, the inlet pipe 1910. is connected to a discharge pipe 1910C for backwash water through a switching valve 1925 on the downstream side of the pump 1923. The circulation pipe 1912 serving as a cleaning liquid path is also branched. A branch pipe 1912A is connected to the oil-containing wastewater storage tank 1921, and a branch pipe 1912B is connected to the cleaning liquid storage tank 1922. Furthermore, by inserting a switching valve 1929 at the branching position, a non-filtered, untreated liquid is returned to the oil-containing wastewater storage tank 1921, and the alkaline cleaning liquid is returned to the cleaning liquid storage tank 1922 for circulation during alkali cleaning,

The outlet pipe 198 which is connected to the outlet port 197a for filtered, treated liquid SL and takes out treated liquid is connected to a treated liquid storage tank 1926. At the same time, since the treated liquid stored in the treated liquid storage tank 1926 is used as backwash water, a backwash pipe 1928 inserted with a backwash pump 1927 is connected between the treated liquid storage tank 1926 and the outlet pipe 198. A diffusion air inlet pipe 1914 is connected to a blower 1915 inserted into the pipe 1910 close to the inlet port 1936a of the separation membrane module 1931, and thereby, diffusion air is fed into the hollow fiber membranes 1932.

The asymmetric membrane is also suitable for membrane distillation. The method of membrane distillation includes passing a heated vaporizing stream of a liquid through a porous membrane, whereby a vapor of the liquid flows via the pores of the membrane to the other side of the membrane, and condensing the vapor on the other side of the membrane to give a distillate stream. An embodiment of a module for membrane distillation is shown in FIG. 14. As shown in FIG. 14, the module is divided into a cold side C and a hot side H. A relatively cold feed stream 211 is pumped with feed pump 1P into the parallel feed channels 212, at the cold side C. These feed channels are constructed by the non-porous walls 213 and a spacer material 214. In these feed channels 212, the feed stream is heated by taking up heat from the warmer distillate channels 215 at the other side of the walls 213. So, gradually the feed stream becomes hotter and leaves the module as stream 216 at the hot side H, with the aid of pump 6P. This pump ensures by suction that the pressure in the feed channels 212 is relatively low; typically between 0.1 and 3.0 bar (absolute pressure). The relatively hot feed stream 216 is pumped into a heat exchanging device 217, where it is heated further by an external heat input 2115, (the heat used can be waste heat, solar heat, steam, hot solid material, etc.), and leaves the device as relatively hot retentate stream 218. Stream 218 enters the module at the hot side H, and flows through the parallel placed retentate channels 219 in more or less counter-current flow with stream 211. The retentate channels 219 are constructed by the porous asymmetric membranes 2110 and the spacer material 2111. In these retentate channels, the retentate stream 218 gradually becomes cooler because of evaporation of water vapor, and some heat conduction, through the porous asymmetric membranes 2111 into the distillate channels 215, where the water vapor condenses forming a pure, liquid distillate 2113. The distillate channels are delimited by the porous asymmetric membrane 2110 at one side and a non-porous condenser wall 213 at the other side. Inside channels 215, a spacer material 2112 can be optionally disposed. The released heat in the distillate channels 215 is primarily transferred through walls 213 into the feed stream 211, flowing in the feed channels 212. The liquid distillate leaves the module preferably at the cold side C, by which also heat of stream 2113 is recovered and transferred into stream 211. It can, however, also be discharged at both sides C and H of the module. This discharge can take place by gravity, pumping, and/or by pressure build up inside channels 215 as a result of the water produced. The relatively cool and concentrated retentate stream leaves the module at the cold side C as stream 2114. For a large part of the module, especially the hot side H, the absolute liquid pressure inside the retentate channels 219 is higher than in the corresponding feed channels 212. The absolute liquid pressures in the retentate channels can range between 1.0 and 4.0 bar, for example.

In addition, the module is useful for separating gases and/or vapors from mixtures of liquids or mixtures of liquids gases using the membrane separation processes of membrane stripping, membrane distillation. In membrane stripping, a material permeating through or across the membrane is removed from the module as a gas or a vapor. In membrane distillation, a membrane is used and the material permeating through or across the membrane is condensed and removed from the device as a liquid.

The invention includes at least the following embodiments.

Embodiment 1. A porous membrane, wherein the porous membrane comprises, consists essentially of, or consists of: a poly(phenylene ether) copolymer comprising, consisting essentially of, or consisting of first and second repeat units having structure (I):

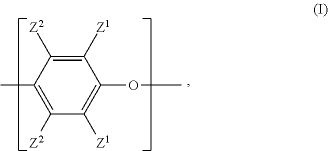

wherein each occurrence of Z¹ is independently halogen, unsubstituted or substituted C₁₋₁₂ hydrocarbyl provided that the hydrocarbyl group is not tertiary hydrocarbyl, C₁₋₁₂ hydrocarbylthio, C₁₋₁₂ hydrocarbyloxy, or C₂₋₁₂ halohydrocarbyloxy, wherein at least two carbon atoms separate the halogen and oxygen atoms, wherein each occurrence of Z² is independently hydrogen, halogen, unsubstituted or substituted C₁-C₁₂ hydrocarbyl provided that the hydrocarbyl group is not tertiary hydrocarbyl, C₁₋₁₂ hydrocarbylthio, C₁₋₁₂ hydrocarbyloxy, or C ₂₋₁₂ halohydrocarbyloxy, wherein at least two carbon atoms separate the halogen and oxygen atoms; wherein the first and second repeat units are not the same; and wherein the porous membrane has at least one of a molecular weight cut off of less than 40 kilodaltons and a surface pore size of 0.001 to 0.1 micrometers.

Embodiment 2 . The porous membrane of embodiment 1, comprising 20 to 100 weight percent of the poly(phenylene ether) copolymer.

Embodiment 3 . The porous membrane of embodiment 1 or 2, wherein the poly(phenylene ether) copolymer comprises: 100 to 20 mole percent repeat units derived from 2,6-dimethylphenol; and 0 to 80 mole percent repeat units derived from a second monohydric phenol (II) wherein Z is C₁₋₁₂, alkyl, C₃₋₁₂, cycloalkyl, or monovalent group (III)

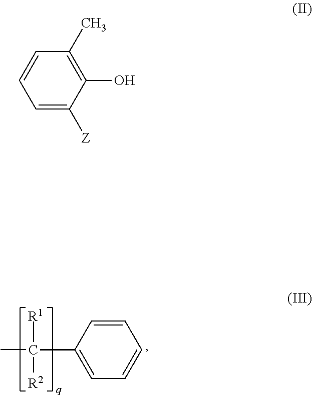

wherein q is 0 or 1, and R¹ and R² are independently hydrogen or C₁₋₆ alkyl; wherein all mole percents are based on the total moles of all repeat units; and wherein the poly(phenylene ether) copolymer block has an intrinsic viscosity of 0.1 to 1.5 deciliters per gram, measured in chloroform at 25° C.

Embodiment 4 . The porous membrane of any of embodiments 1-3, wherein the copolymer has an intrinsic viscosity of 0.7 to 1.5 deciliters per gram, when measured in chloroform at 25° C.

Embodiment 5 . The porous membrane of embodiment 3 or 4, wherein the copolymer comprises: 80 to 20 mole percent repeat units derived from 2,6-dimethylphenol; and 20 to 80 mole percent repeat units derived from the second monohydric phenol.

Embodiment 6 . The porous membrane of any of embodiments 3-5, wherein the second monohydric phenol is 2-methyl-6-phenylphenol.

Embodiment 7 . The porous membrane of any of embodiments 1-6, further comprising poly(2,6-dimethyl-1,4-phenylene ether), polyethersulfone, polysulfone, polyphenylsulfone, or a combination comprising at least one of the foregoing.

Embodiment 9 . A method of making the porous membrane of any of embodiments 1-7, comprising dissolving the poly(phenylene ether) copolymer in a water-miscible polar aprotic solvent to form a porous membrane-forming composition; phase-inverting the porous membrane forming-composition in a first non-solvent to form the porous membrane; optionally washing the porous membrane in a second non-solvent; and optionally drying the porous membrane.

Embodiment 10 . A porous membrane-forming composition for forming the porous membrane of any of claims 1-7, comprising the poly(phenylene ether) copolymer and a water-miscible polar aprotic solvent, wherein the poly(phenylene ether) copolymer is dissolved in the water-miscible polar aprotic solvent.

Embodiment 11 . The method of embodiment 9 or the porous membrane-forming composition of embodiment 10, wherein the solubility of the poly(phenylene ether) copolymer in the water-miscible polar aprotic solvent is 50 to 400 grams per kilogram at 25° C., based on the combined weight of the poly(phenylene ether) copolymer and the solvent.

Embodiment 12 . The porous membrane of any of embodiments 1-7, the porous membrane formed by the method of embodiment 9, or the porous membrane formed from the porous membrane-forming composition of embodiment 10, wherein the porous membrane is in a sheet, disc, spiral wound, plate and frame, hollow fiber, capillary, or tube configuration.

Embodiment 13 . The porous membrane of any of embodiments 1-7, the porous membrane formed by the method of embodiment 9, or the porous membrane formed from the porous membrane-forming composition of embodiment 10, wherein the membrane is a porous flat sheet.

Embodiment 14 . The porous membrane of any of embodiments 1-7, the porous membrane formed by the method of embodiment 9, or the porous membrane formed from the porous membrane-forming composition of embodiment 10, wherein the porous membrane is a porous hollow fiber.

Embodiment 15 . A separation module comprising the porous membrane of any of embodiments 1-7, the porous membrane formed by the method of embodiment 9, the porous membrane formed from the porous membrane-forming composition of embodiment 10, or the porous membrane of any of embodiments 12-14.

Embodiment 16 . The separation module of embodiment 15, wherein the separation module is adapted for dead-end filtration, outside-in filtration, inside-out filtration, or cross-flow filtration.

Embodiment 17 . The separation module of embodiment 15, wherein the separation module is selected from a microfiltration module, a nanofiltration module, an ultrafiltration module, a reverse osmosis module, and a membrane distillation module.

Embodiment 18 . The separation module of embodiment 15, comprising a bundle of asymmetric hollow fibers.

Embodiment 19 . The separation module of embodiment 18, wherein the bundle of hollow fibers are disposed within an enclosure configured for fluid separation.

Embodiment 20 . The separation module of embodiment 18 or 19, wherein the separation module comprises an enclosure configured to contain the bundle of asymmetric hollow fibers, the enclosure having an outlet configured for withdrawing a permeate fluid; a first encasement comprising a thermoset or a thermoplastic polymeric material and located at a first end of the bundle, arranged such that the hollow fiber membranes are embedded in the first encasement and communicate through the first encasement and are open on an outer face of the first encasement; a second encasement comprising a thermoset or a thermoplastic polymeric material and located at a second end of the bundle opposite the first end of the bundle, arranged such that the hollow fiber membranes are embedded in the second encasement and communicate through the second encasement and are open on an outer face of the second encasement; a first end cap arranged and configured for attaching and sealing to the first end of the bundle or enclosures at or near the first encasement; a second end cap arranged and configured for attaching and sealing to the second end of the bundle or enclosures at or near the second encasement; an inlet for introducing a fluid mixture to be separated into bores of the hollow fiber membranes at the first encasement; and an outlet for withdrawing a retentate fluid from the bores for the hollow fiber membranes at the second encasement.

Embodiment 21 . The separation module of any of embodiments 18-20, comprising a plurality of the bundles of asymmetric hollow fibers.

Embodiment 22 . The separation module of any of embodiments 15-17, wherein separation module comprises: a hollow core comprising perforations; the porous composite membrane wound around the core; and a spacer disposed adjacent the porous composite membrane.

Embodiment 23 . The separation module of any of embodiments 15-17 or 22, further comprising at least one of an inner spacer and an outer spacer adjacent the porous composite membrane.

Embodiment 24 . A separation module comprising a spiral wound porous flat sheet of embodiment 13.

Embodiment 25 . A separation module comprising 10 to 10,000 of the porous asymmetric hollow fibers of embodiment 14.

Embodiment 26 . A method of filtration comprising passing a feedstream through the separation module of any of embodiment 15-25 such that it contacts a first side of the porous composite membrane, and passing a permeate through the porous composite membrane to provide a permeate stream and a concentrated feedstream.

Embodiment 27 . The method of embodiment 26, wherein the method comprises countercurrent flow distribution.

Embodiment 28 . A dialysis device for conducting hemodialysis on a patient suffering from liver failure, the device comprising the porous membrane of any of embodiments 1-7 or 12-14, or the porous membrane formed by the method of embodiment 9 or from the membrane-forming composition of embodiment 10.

Embodiment 29 . The dialysis device of embodiment 28, wherein the dialysis device comprises the separation module of any of embodiments 15-25.

Embodiment 30 . The dialysis device of embodiment 29, wherein the porous membrane allows the passage of molecules having a molecular weight of up to 45 kilodaltons with a sieving coefficient of 0.1 to 1.0 in the presence of whole blood; wherein the dialysis device reduces the concentration of protein-bound toxins and inflammatory cytokines in the blood of the patient; wherein the dialysis device reduces the concentration of unconjugated bilirubin and bile acids in the blood of the patient; and wherein the dialysate passing the said dialysis membrane comprises from 1% to 25% human serum albumin.

Embodiment 31 . A method of dialysis, the method comprising passing blood through the separation module of any of embodiments 15-25 such that it contacts a first side of the porous membrane, and passing a dialysis solution through the separation module such that it contacts a second opposite side of the porous membrane to remove waste products from the blood.

Embodiment 32 . A method for the treatment of liver failure, the method comprising conducting hemodialysis on a patient suffering from liver failure using a liver dialysis device comprising the porous membrane of any of embodiments 1-7 or 12-14, or the porous membrane formed by the method of embodiment 9 or from the composition of embodiment 10.

Embodiment 33 . The method for the treatment of liver failure of embodiment 32, wherein the dialysis device comprises the separation module of any of embodiments 15-25.

Embodiment 34 . A method of sugar purification, the method comprising passing a fluid comprising a combination of polysaccharides through the separation module of any of embodiments 15-25 such that the fluid contacts a first side of the porous membrane, and passing a polysaccharide through the membrane to purify the sugar.

Embodiment 35 . A method of protein or enzyme recovery comprising: passing a fluid comprising a protein or enzyme through the separation module of any of embodiments 15-25 such that the fluid contacts a first side of the porous composite membrane; and removing a component from the fluid by passing the component through the membrane to provide a retentate stream enriched in the protein or enzyme to recover the protein or enzyme.

Embodiment 36 . A method of water purification comprising passing a feedwater through the separation module of any of embodiments 15-25 such that the feedwater contacts a first side of the porous composite membrane with a pressure greater than osmotic pressure to produce purified water.

Embodiment 37 . A water pretreatment system comprising the separation module of any of embodiments 15-25.

Embodiment 38 . The method of water pretreatment of embodiment 37, wherein the separation module further comprises: a semi-permeable membrane unit for treating a filtrate of the separation module; a back-pressure washing unit for feeding water from a secondary side of the porous membrane to a primary side thereof; a device for feeding a chlorine agent to the water; and a device for feeding an ammoniacal compound and/or an amino group-containing compound to the primary side of the separation module.

Embodiment 39 . A method of pretreating water, the method comprising: receiving feed water; separating the feed water into a concentrator feed and a slipstream; processing the concentrator feed in a concentrator comprising the porous membrane of any of embodiments 1-7 or 12-14, or the porous membrane formed by the method of embodiment 9 or from the composition of embodiment 10, to generate a hypertonic solution; combining the slipstream and the hypertonic solution to generate an effluent capable of decomposition into purified water and a recirculating hypertonic solution.

Embodiment 40 . The method of embodiment 39, wherein the concentrator comprises the separation module of any of embodiments 15-25.

Embodiment 41 . A blood oxygenator comprising: a housing; a plurality of hollow fibers comprising the porous membrane of any of embodiments 1-7 or 12-14, or the porous membrane formed by the method of embodiment 9 or from the composition of embodiment 10, disposed within the housing for transporting a first fluid therethrough; a first inlet in fluid communication with the fibers for delivering the first fluid thereto; a first outlet in fluid communication with the fibers for receiving the first fluid therefrom; a second inlet and a second outlet in communication with regions disposed exteriorly of the hollow fibers.

Embodiment 42 . The blood oxygenator of embodiment 41, wherein the porous membrane is contained within the separation module of any of embodiments 15-25.

Embodiment 43 . The blood oxygenator of embodiment 42, wherein the first fluid is blood, and wherein the second fluid is an oxygen-containing gas.

Embodiment 44 . The blood oxygenator of embodiment 43, wherein the first fluid is blood, and wherein the second fluid is a liquid which comprises molecular oxygen.

Embodiment 45 . A separation module for oil-containing wastewater treatment, which separates water-insoluble oil from oil-containing wastewater, the separation module comprising the porous membrane of any of embodiments 1-7 or 12-14, or the porous membrane formed by the method of embodiment 9 or from the composition of embodiment 10.

Embodiment 46 . A system for oil-containing wastewater treatment comprising the separation module of embodiment 45.

Embodiment 47 . A method of oil-containing wastewater treatment comprising treating an oil-containing wastewater with the separation module of embodiment 46.

Embodiment 48 . The method of embodiment 47 further comprising directing a cleaning liquid comprising an alkaline aqueous solution to a surface of the porous membrane to remove water-insoluble oil adhering to the surface of the porous membrane of the separation membrane module.

Embodiment 49 . An ultrafiltration device comprising: a bundle of tubular or capillary membranes comprising the porous membrane of any of embodiments 1-7 or 12-14, or the porous membrane formed by the method of embodiment 9 or from the composition of embodiment 10; and a filter housing for the bundle of tubular or capillary membranes, the filter housing comprising an inlet and an outlet; wherein the tubular or capillary membranes are permanently hydrophilic and pore size decreases in the direction of the liquid flow; and wherein the bundle of tubular or capillary membranes are open at a first inlet end and sealed at the other end, and are held at the first inlet end in a membrane holder which closes off the space between the bundles tubular or capillary membranes and the filter housing.

Embodiment 50 . An apparatus for purification of a liquid by membrane distillation comprising: a feed channel; a distillate channel; and a retentate channel, wherein the distillate channel and retentate channel are separated by the porous membrane of any of embodiments 1-7 or 12-14, or the porous membrane formed by the method of embodiment 9 or from the composition of embodiment 10.

Embodiment 51 . The apparatus for purification of a liquid by membrane distillation of claim 50, wherein the apparatus comprises: a segment comprising a first distribution chamber for a feed liquid to be supplied, a second distribution chamber located opposite the first distribution chamber for feed liquid to be discharged, a third distribution chamber for retentate stream to be supplied and a fourth distribution chamber opposite the third distribution chamber for the retentate stream to be discharged, whereby the segment is provided with a first pump for pumping the feed stream pressure into the segment and a second pump which is arranged downstream the second distribution chamber for pumping the retentate stream under pressure into the retentate channel, the wall between the feed channel and the distillate channel comprises a condenser surface in the form of a non-porous membrane, and the wall between the retentate channel and the distillate channel comprises the porous membrane, and wherein inside the retentate channel a further channel is arranged for allowing a fluid stream to be brought into heat transfer contact with the retentate stream.

The invention is further illustrated by the following non-limiting examples.

### PREPARATIVE EXAMPLES

### Synthesis of MPP-DMP Copolymers

The preparation, characterization and properties of poly(phenylene ether)s has been described by G. Cooper and J. Bennett in Polymerization Kinetics and Technology, Vol. 128, pp. 230-257, Jun. 1, 1973 (ACS Adv. in Chem. Series). MPP-DMP copolymers were prepared by dissolving the monomers in toluene and conducting oxidative copolymerization with copper-diamine catalyst complexes in the presence of oxygen. The copolymerizations were conducted in a bubbling polymerization reactor equipped with a stirrer, temperature control system, nitrogen padding, oxygen bubbling tube, and computerized control system. The reactor was also equipped with a feeding pot and pump for dosing reactants into the reactor. When the desired degree of polymerization was achieved, the flow of oxygen was stopped and the copper was removed by liquid-liquid extraction with a water-soluble chelating agent. The DMP-MPP copolymers were recovered via non-solvent precipitation by pouring the toluene solution into an excess of methanol with vigorous stiffing followed by drying in an oven at 120° C. under a stream of dry nitrogen. Glass transition temperatures (Tg) were determined using differential scanning calorimetry (DSC). The molecular weight distributions of the polymers were characterized via size-exclusion chromatography methods employing chloroform as the mobile phase and calibration against a polystyrene standard. Alternatively the degree of polymerization was characterized by measurement of intrinsic viscosity (IV) in CHCl₃ using the Ubbelohde method.

TABLE 3
Characterization of MPP-DMP Copolymers of Preparative
Examples 1-4 and 11-13

| | | | | | IV in | |
|---|---|---|---|---|---|---|
| Ex. | MPP/DMP | GPC Mn | GPC Mw | GPC D | CHCl₃ | Tg |
| No. | (mole/mole) | (g/mole) | (g/mole) | (Mw/Mn) | (dL/g) | ° C. |
| 1 | 50/50 | 20,213 | 219,130 | 10.8 | 0.83 | 185 |
| 2 | 20/80 | 50,310 | 172,100 | 3.4 | 1.04 | 210 |
| 3 | 50/50 | 39,820 | 194,900 | 4.9 | 0.97 | 187 |
| 4 | 80/20 | 22,620 | 241,000 | 10.7 | 0.96 | 177 |
| 11 | 20/80 | 63,010 | 210,800 | 3.3 | 1.14 | — |
| 12 | 50/50 | 42,460 | 216,200 | 5.1 | 0.98 | — |
| 13 | 80/20 | 36,490 | 310,700 | 8.5 | 1.08 | — |

### Examples 5-10

### General Procedure for Casting Membranes Via Solvent/Non-Solvent Phase Inversion Process

The polymer was dissolved in chromatography grade NMP totaling 8-10 g in a 20-mL glass vial, sealed tightly, and placed on a low speed roller for 13-48 hr. until a homogenous solution was formed. The solution was poured in an oblong puddle and an adjustable height doctor blade was used to drag across the glass plate at a constant speed by hand. The entire glass plate bearing the cast polymer film was fully submerged into an initial non-solvent bath (25-100 wt. % DI water in NMP) until the membrane begins to lift off the plate. The membrane was transferred off of the glass plate into the intermediate non-solvent bath of 100 wt. % DI water and weighed down at the corners with glass stoppers to allow the exchange of NMP into the water. After 15-45 min. the membrane was transferred to a final non-solvent bath of 100 wt. % water overnight to fully solvent exchange the NMP. The membrane was dried at room temperature. Characterization was performed on pieces cut from the center and most uniform portion of the membrane. The viscosity of the copolymer solutions in NMP was measured at 20° C. using a Brookfield RDV-II Pro viscometer equipped with a small-sample adapter and cylindrical spindle.

### Characterization of Membranes

The surface porosities and cross-sectional morphologies of the membranes were characterized using Carl Zeiss Supra VP scanning electron microscopy (SEM). The “top” membrane surfaces (those that were first in contact with the NMP/water bath) were imaged for selective surface morphology. The membrane samples were coated with˜0.3 nm Pt/Pd target using Cressington 208 high resolution sputter coater equipped with thickness controller MTM-20. The surface morphology was imaged using low voltage capability (≦5 kV, probe current 200 nA and inlens surface sensitive detection mode at 100,000× magnifications. A minimum of 3 images were combined for digital image analysis using Clemex Vision PE 6.0.035 software to estimate the pore size distributions and pooled for the analysis. Samples for cross-sectional imaging were soaked in ethanol for 5 min. and cryo-fractured using liquid nitrogen, then allowed to come to room temperature and dried in air. The cryo-fractured membrane samples were coated with Pt/Pd target and imaged using SEM for cross sectional morphology.

The interaction of the membrane surfaces with water was quantified via measurement of contact angle using a Kruss DA-25 drop shape analysis system. A small square section of membrane was cut from the center of the membrane, and mounted on a glass microscope slide using double-sided tape. A 2-μL water droplet was deposited on the surface. Drop shape was measured using digital curve fitting 5 times with a 1 sec. spacing. Resulting contact angles of the water droplet with the membrane surface were averaged.

### Examples 18-20 and Comparative Example 3

### Hollow Fiber Spinning

The membrane-forming compositions of Ex, 18-20, (containing the MPP-DMP copolymers of Ex, 11-13, respectively) and Comp. Ex, 3 (6020P, PVP K30, and PVP K90) were fabricated into hollow fiber membranes on a laboratory scale by dry-wet immersion precipitation spinning according to the method disclosed in WO2013/131848, using the apparatus shown in FIG. 3, ULTRASON™ E 6020P was maintained for 24 hr, under vacuum prior to mixing to remove all moisture. Mixing was done until homogenous solutions were obtained. The membrane-forming the composition was filtered through a 25-μm metal mesh to remove any residual particles in the composition and degassed for 24 hr, before use. A bore solution of 70 wt. % deionized water and 30 wt. % NMP was prepared and degassed for 24 hr. before use. The copolymer solution along with the bore liquid were simultaneously pumped through a double orifice spinneret and after passing the air gap, and immersed into the coagulation bath. The take-up velocity was controlled by a pulling wheel, which enabled also stretching of the fiber.

A summary of the fiber spinning conditions, spinneret geometry, and measured dimensions of the dried hollow fibers is shown in Table 10. For Comp. Ex. 3, the rinsing bath was held at 65°C. according to the example in the ′848 application, which is understood to be for rinsing away excess PVP from the surface of the hollow fiber. For Examples 18-20, which were prepared from the 20/80, 50/50, and 80/20 MPP-PPE copolymers, respectively, the rinsing bath was held at 30°C. for safety in handling the fibers and because there is no PVP to be washed away. The take-up velocity was adjusted such that the wall thickness of the two hollow fiber samples was in the range of 40-60 μm. The post treatment process for the hollow fiber produced was as described in the ′848 application. The fibers were washed in 70° C. purified water for 3 hr. After 1.5 hr, the water was exchanged. Afterwards the fibers were rinsed for another 24 hr. in water at tap temperature. After the rinsing step, the fibers were hung in the lab to dry in air at ambient temperature.

Based on the finding that the membrane-forming polymer solution viscosity in NMP was very sensitive to the amount of MPP co-monomer in the copolymer, the concentration of each resin was adjusted so as to yield art essentially constant solution viscosity of just over 3,000 cP, As a result there is a direct correlation between the level of MPP co-monomer in the copolymer and the mass of PPE per unit length of fiber, with Ex. 18 demonstrating the most efficient use of resin under the same spinning conditions. The fiber wall thickness was also maintained to a greater extent in Ex. 19, suggesting that with further optimization of fiber spinning conditions io reduce the wall thickness, a greater reduction in mass per unit length can be realized.

TABLE 10
Summary of Process Conditions for Hollow
Fiber Spinning and Fiber Properties

| Example | Comp. Ex. 3 | Ex. 18 | Ex. 19 | Ex. 20 |
|---|---|---|---|---|
| Wt % resin in NMP casting | 14% | 18% | 14% | 20% |
| dope | 6020P^{a} | Ex. 12 | Ex. 11 | Ex. 13 |
| Viscosity (cP at 35° C.) | | 3270 | 3091 | 3137 |
| Dope temp. [° C.] | 35 | 35 | 35 | 35 |
| Die temp. [° C.] | — | — | — | — |
| Shaft temp. [° C.] | ~22 | ~30 | ~30 | ~22 |
| Shaft humidity [%] | 50 | 60 | 60-65 | 60 |
| Room humidity [%] | 35 | 40 | 40 | 40 |
| 1^{st} bath temp. [° C.] | 30 | 30 | 30 | 30 |
| 2^{nd} bath temp. [° C.] | 65 | 30 | 30 | 30 |
| Air Gap [cm] | 100 | 100 | 100 | 100 |
| Dope extrusion rate [mL/min] | 1.56 | 1.56 | 1.56 | 1.56 |
| Bore extrusion rate [mL/min] | 3.1 | 3.1 | 3.1 | 3.1 |
| Take up velocity [m/min] | 9.12 | 7.04 | 7.07 | 7.00 |
| Spinneret dimensions | | | | |
| Inner diameter [mm] | 0.4 | 0.4 | 0.4 | 0.4 |
| Outer diameter [mm] | 1.12 | 1.12 | 1.12 | 1.12 |
| Dry hollow fiber dimensions | | | | |
| by SEM | | | | |
| Inner diameter [μm] | 445 | 605 | 510 | 605 |
| Wall thickness [μm] | 59 | 41 | 47 | 23 |
| Mass per km (g) | 25.9 | 40.2 | 31.1 | 43.3 |

^{a}Plus 5% PVP K30, 2% PVP K90, and 3% H₂O

### Preparation of Hollow Fiber Membrane Modules

Lab scale hollow fiber membrane modules were prepared for the clean water flux and molecular weight cut off measurements. 5-10 fibers, depending on the geometry were guided through polypropylene tubes and the t-connections, which provide access to the outer surface of the hollow fibers. Both ends were -sealed with hot glue. After the glue hardened, the modules were carefully cut open at one or both ends to expose the inner core of the hollow fibers to make them ready to use. The membrane length was between 25 and 30 cm. The fibers of Ex. 20 were more brittle than the other fibers, and extra care was required to glue the fibers of Ex. 20 into the modules to avoid damaging the fibers.

### Measurement of Clean Water Flux

Clean water flux (CWF) was measured as follows. A pump was connected to a mass flow controller and a pressure sensor. Behind the pressure sensor the membrane module was connected so that the filtration direction was inside-out, that is the water was forced into the bore side of the membrane and permeated through the membrane to the outside of the membrane. The filtration mode was dead end filtration, that is only one end of the filtration module was cut open and connected to the feed solution. The flow rate was set to 100 g/h and the feed pressure was recorded over time. After the pretreatment of the membrane modules, the experiment was run for 1 hr. to achieve steady state conditions.

Prior to the measurement, all the hollow fibers were wetted with a mixture of 50 wt. % water and 50 wt. % ethanol. Afterwards clean water was permeated through the hollow fiber membranes for 15 minutes to remove all residual ethanol from the fibers. The measurement was started directly after the pretreatment. The results of the water flux measurements are provided in Table 11.

TABLE 11
Clean Water Flux Measurements

| Ex. | Clean Water Flux (L/(h · m² · bar)) |
|---|---|
| CE3 (PES/PVP) | |
| Module 1 | 8.0 |
| Module 2 | 8.6 |
| Module 3 | 7.9 |
| Module 4 | 9.1 |
| Average | 8.4 ± 0.6 |
| E18 (E12 - 20/80 MPP-DMP) | |
| Module 1 | 44.3 |
| Module 2 | 24.9 |
| Module 3 | 64.8 |
| Module 4 | 60.1 |
| Module 5 | 54.4 |
| Average | 49.7 ± 15.8 |
| E19 (E11 -50/50 MPP-DMP) | |
| Average of 4 Modules | 40.2 ± 21 |
| E20 (E13 - 80/20 MPP-DMP) | |
| Average of 3 Modules | 133 ± 18.5 |

As can be seen from Table 11, the highest clean water flux (133 L/(h·m²·bar)) was obtained at the highest MPP comonomer content—the 80/20 MPP-DMP copolymer of Ex. 20. Without being bound by theory, this effect may be due to the thinner fiber cross-section obtained with those fibers—a wall thickness of only 23 μm, as reported in Table 10. Although the individual values vary, the clean water flux for all the PPE copolymer fibers (Ex. 18-20) are substantially higher than the C. Ex. 3 fiber, which has a clean water flux of about 8 L/(h·m²·bar), and which was taught by prior art application publication ′848.

### Measurement of Molecular Weight Cut-Off

Prior to the measurement of the molecular weight cut-off (MWCO), all membrane modules were wetted with a mixture of 50 wt. % water and 50 wt. % ethanol. Next, clean water was permeated through the hollow fiber membranes for 15 minutes to remove all residual ethanol from the fibers. The measurement was started directly after the pretreatment.

Both ends of the hollow fiber filtration modules were cut and the feed solution was pumped through the inside of the hollow fibers and the retentate recirculated to the feed tank. The permeate solution is circulated across the outside of the fibers via the T-connectors and recycled to a separate feed tank. The cross flow velocity was controlled via the pump and the feed, retentate, and pressure are recorded. The permeate pressure was at ambient pressure. A valve at the retentate side can optionally be used to control the retentate pressure.

A turbulent flow inside the hollow fiber is desirable in order to prevent concentration polarization during the experiment. To provide turbulent flow, the cross flow velocity is set to target a Reynolds number of about 3000. The Reynolds number is defined according to Equation 1, whereas “η” is defined as the dynamic viscosity of the fluid, “ρ” is defined as the density of the fluid, “v” defined as the fluid velocity and “d” defined as the inner fiber diameter.

$$Re = \frac{ρ * v * d}{η} \qquad ( Eq . 1 )$$

As a feed solution, a mixture of four different dextrans, which differ in molecular weight (1 kDa, 4 kDa, 8 kDa and 40 kDa), was used. The concentration in the feed solution was 0.5 g/L for each dextran. The molecular weight cut off is defined as that molecular weight of a species which is retained up to 90 percent by the membrane. The retention is calculated by comparing the gel permeation chromatography of the initial solution of dextrans to that measured on permeate and retentate solutions after reaching equilibrium.

Three filtration modules of each of Comparative Example 3 and Examples 18-20 were tested, and the results are summarized in Table 12. For the three PES modules of Comparative Example 3, it was possible to run the MWCO experiment under conditions of a Reynolds number (Re) of 3000. However, no MWCO was determined for two modules (Retention was always below 90 percent for the given feed.) and for the third module the MWCO was not stable over time.

In contrast to the PES/PVP hollow fibers of Comparative Example 3, the PPE copolymer hollow fibers of Examples 18-20 appeared to be defect-free under the same conditions of high Re (3,000-3,600) and high trans-membrane pressure (TMP, 1.9-3.5 bar) and yielded stable MWCO values of 6-15 kDa. Thus the membranes of Examples 18-20 provide an improved combination of higher CWF and stable low MWCO over the membrane produced from PES and PVP. In addition, the membranes of Ex. 18-20 provided improved mechanical integrity. The fact that this performance can be achieved from membranes formed from inherently hydrophobic PPE resin in the absence of pore-forming additives (hydrophilic polymer), using only a simple wetting process based on aqueous ethanol, is surprising.

Stable readings were readily obtained for the additional examples: since the MWCO values at either extreme of MPP co-monomer content were essentially the same we conclude that there is no significant effect of this parameter on the ability to form well-controlled pore size distributions from the PPE during hollow fiber spinning

TABLE 12
Molecular Weight Cut-off Measurements

| Hollow | | MWCO (kDa) | | | |
|---|---|---|---|---|---|
| Fiber | Polymer | 60 min | 75 min | 120 min | 180 min |

| | | |
|---|---|---|
| CE3 | CE2 (PES/PVP) | Re = 3,000; flow = 100 L/h; |
| | | TMP = 2.1 bar |

| | | | |
|---|---|---|---|
| 10.1 | — | 44.3 | 59.3 |

| | | |
|---|---|---|
| | | 90 Percent retention was not reached. |
| | | 90 Percent retention was not reached. |
| E18 | E12 (50/50 MPP-DMP) | Re = 3,000; Flow = 140 L/h; |
| | | TMP = 2.1 bar |

| | | | |
|---|---|---|---|
| 8.3 | 7.3 | 6.3 | — |
| 5.2 | 6.6 | 5.3 | — |
| 6.4 | 5.2 | 5.2 | — |

| | | |
|---|---|---|
| | | Average = 5.6 |
| E19 | E11 (20/80 MPP-DMP) | Re = 3,600; Flow = 140 L/h; |
| | | TMP = 1.9 bar |

| | | | |
|---|---|---|---|
| 61.7 | 54.5 | 51.4 | — |
| 15.9 | 14.6 | 13.6 | — |
| 12.8 | 13.6 | 13.4 | — |

| | | |
|---|---|---|
| | | Average = 13.5 |
| E20 | E13 (80/20 MPP-DMP) | Re = 3,250; Flow = 150 L/h; |
| | | TMP = 3.5 bar |

| | | | |
|---|---|---|---|
| 16.3 | — | 16.1 | 15.6 |
| 14.0 | — | 13.5 | 17.5 |
| 17.7 | — | 19.5 | 13.2 |

Average = 15.4

### Summary of Hollow Fiber Spinning

The results of the hollow fiber spinning trials (Ex. 18-20) illustrate that MPP-DMP copolymers that incorporate the minimum amount of MPP co-monomer required for solubility in solvents such as NMP, for example the 20/80 MPP-DMP copolymer of Ex. 11, also result in the maximum increase in solution viscosity for a given concentration of copolymer. The results also show that PPE copolymers having much less than 50 mole % MPP comonomer provide an advantageous reduction in the mass of resin per unit length of hollow fiber, for example, 31.1 km/g for the hollow fibers of Ex. 19 fabricated from 20/80 MPP-DMP copolymer of Example 11. The hollow fibers of Examples 18-20 show that MPP-DMP copolymers having a weight average molecular weight of 150,000 to 400,000 Da and a broad molecular weight distribution, with polydispersity values of 3 to 9, provide high-quality hollow fibers. The polymerization process for these copolymers can be scaled up for industrial production. Moreover, weight-average molecular weight of these copolymers can be varied to optimize dope solution viscosity, and surface pore size and distribution.

### Sem Comparison of Flat Sheet and Hollow Fiber Morphology

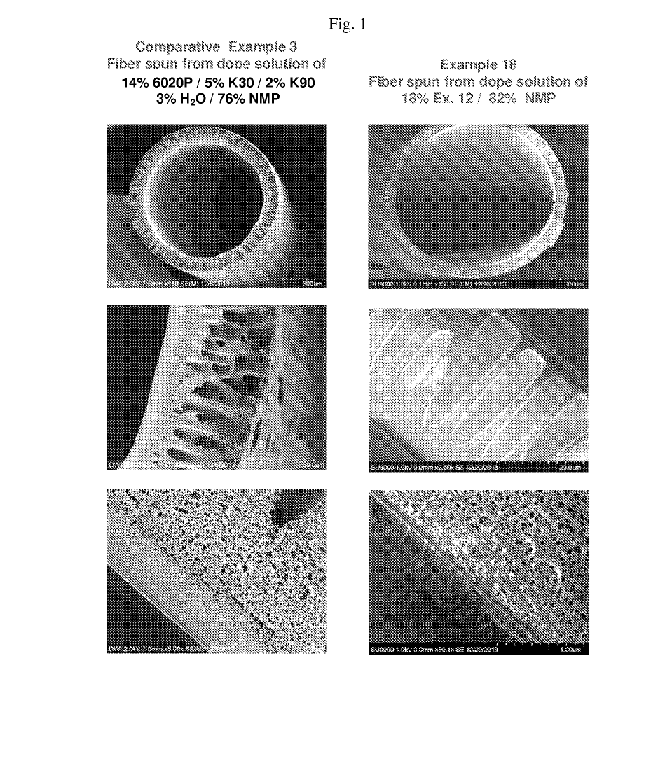

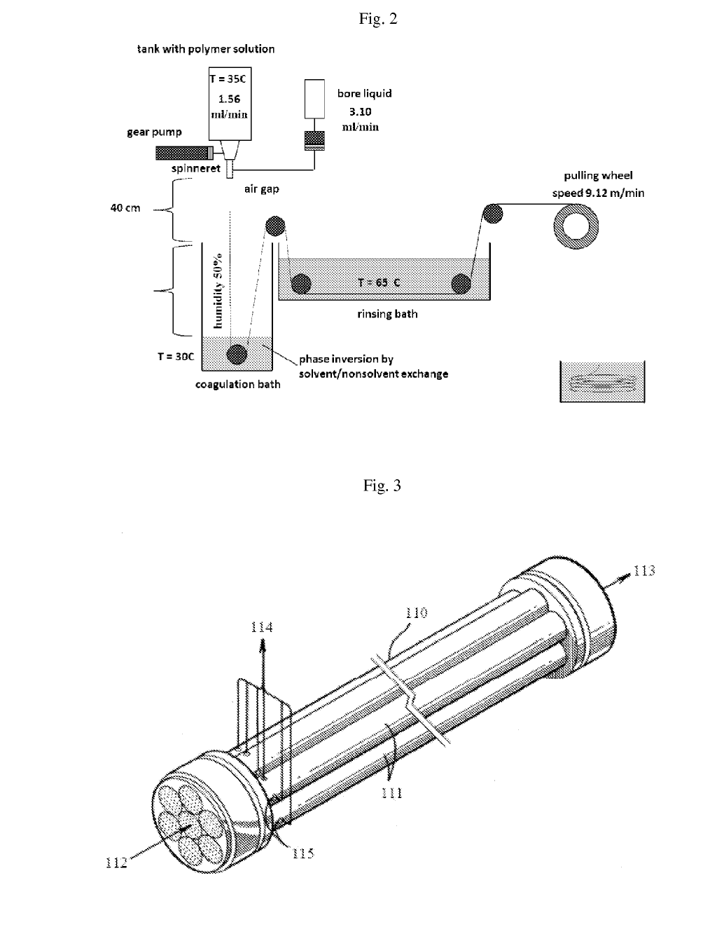

The hollow fibers of Comp. Ex. 3 and Ex. 18 were analyzed by SEM, the results of which are shown in FIG. 2. The hollow fibers of Comp. Ex. 3, prepared from PES/PVP, show a strongly asymmetric cross-sectional morphology, and similar to those obtained for flat membrane castings of the same dope composition. The dense selective layer appears to be thin for the PES/PVP membrane in both flat and the hollow fiber geometries. In comparison, the morphology of the hollow fiber of Ex. 18 shows a dense spongy morphology that persists across the fiber cross-section, which is also consistent with flat membranes produced from the same dope composition. Thus the poly(phenylene ether) copolymers disclosed herein provide membrane-forming characteristics that are superior to those of PES/PVP polymers in both flat and hollow fiber geometries.

The use of the terms “a” and “an” and “the” and similar referents in the context of describing the invention (especially in the context of the following claims) is to be construed to cover both the singular and the plural, unless otherwise indicated herein or clearly contradicted by context. “Or” means “and/or.” The endpoints of all ranges directed to the same component or property are inclusive and independently combinable. Disclosure of a narrower range or more specific group in addition to a broader range is not a disclaimer of the broader range or larger group. All ranges disclosed herein are inclusive of the endpoints, and the endpoints are independently combinable with each other. The terms “first” and “second” and the like, as used herein do not denote any order, quantity, or importance, but are only used to distinguish one element from another. The term “comprises” as used herein is understood to encompass embodiments consisting essentially of, or consisting of, the named elements.

Unless defined otherwise, technical and scientific terms used herein have the same meaning as is commonly understood by one of skill in the art to which this invention belongs. A “combination” is inclusive of blends, mixtures, alloys, reaction products, and the like.

As used herein, the term “hydrocarbyl” refers broadly to a moiety having an open valence, comprising carbon and hydrogen, optionally with 1 to 3 heteroatoms, for example, oxygen, nitrogen, halogen, silicon, sulfur, or a combination thereof. Unless indicated otherwise, the hydrocarbyl group can be unsubstituted or substituted, provided that the substitution does not significantly adversely affect synthesis, stability, or use of the compound. The term “substituted” as used herein means that at least one hydrogen on a hydrocarbyl group is replaced with another group (substituent) that contains a heteroatom selected from nitrogen, oxygen, sulfur, halogen, silicon, or a combination thereof, provided that the normal valence of any atom is not exceeded. For example, when the substituent is oxo (i.e. “═O”), then two hydrogens on a designated atom are replaced by the oxo group. Combinations of substituents and/or variables are permissible provided that the substitutions do not significantly adversely affect the synthesis, stability or use of the compound.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A separation module comprising a porous membrane, wherein the porous membrane comprises a poly(phenylene ether) copolymer having at least one of a molecular weight cut off of less than 40 kilodaltons and a surface pore size of 0.001 to 0.1 micrometers.

2. The separation module of claim 1, wherein the porous membrane is a porous flat sheet.

3. The separation module of claim 2, wherein the porous flat sheet is wound into a spiral.

4. The separation module of claim 1, wherein the porous membrane is a porous hollow fiber.

5. The separation module of claim 1, wherein the porous membrane is a capillary or tubular porous membrane.

6. The separation module of claim 4, wherein the separation module comprises: an enclosure configured to contain a bundle of the porous hollow fibers, the enclosure having an outlet configured for withdrawing a permeate fluid; a first encasement comprising a thermoset or a thermoplastic polymeric material and located at a first end of the bundle, arranged such that the porous hollow fibers are embedded in the first encasement and communicate through the first encasement and are open on an outer face of the first encasement; a second encasement comprising a thermoset or a thermoplastic polymeric material and located at a second end of the bundle opposite the first end of the bundle, arranged such that the porous hollow fibers are embedded in the second encasement and communicate through the second encasement and are open on an outer face of the second encasement; a first end cap arranged and configured for attaching and sealing to the first end of the bundle or enclosures at or near the first encasement; a second end cap arranged and configured for attaching and sealing to the second end of the bundle or enclosures at or near the second encasement; an inlet for introducing a fluid mixture to be separated into bores of the porous hollow fibers at the first encasement; and an outlet for withdrawing a retentate fluid from the bores for the porous hollow fibers at the second encasement.

7. A method of hemodialysis, the method comprising: passing blood through the separation module of claim 1, such that the blood contacts a first side of the porous membrane; and passing a dialysis solution through the separation module such that it contacts a second opposite side of the porous membrane to remove waste products from the blood.

8. A dialysis device for conducting liver dialysis on a patient suffering from liver failure, the device comprising the separation module of claim 1.

9. The dialysis device of claim 8, wherein the porous membrane allows the passage of molecules having a molecular weight of up to 45 kilodaltons with a sieving coefficient of 0.1 to 1.0 in the presence of whole blood; wherein the dialysis device reduces the concentration of protein-bound toxins and inflammatory cytokines in the blood of the patient; wherein the dialysis device reduces the concentration of unconjugated bilirubin and bile acids in the blood of the patient; wherein the dialysate passing the porous membrane comprises from 1% to 25% human serum albumin.

10. A blood oxygenator comprising: a housing; a separation module comprising a plurality of the porous hollow fibers of claim 4 disposed within the housing for transporting a first fluid therethrough; a first inlet in fluid communication with the porous hollow fibers for delivering the first fluid thereto; a first outlet in fluid communication with the porous hollow fibers for receiving the first fluid therefrom; and a second inlet and a second outlet in communication with regions disposed exteriorly of the porous hollow fibers.

11. A method of sugar purification, the method comprising passing a fluid comprising a combination of polysaccharides through the separation module of claim 1, such that the fluid contacts a first side of the porous membrane, and passing a polysaccharide through the porous membrane to purify the sugar.

12. A method of protein or enzyme recovery comprising: urging a fluid comprising a protein or enzyme through the separation module of claim 1, such that the fluid contacts a first side of the porous membrane; and removing a component from the fluid by passing the component through the porous membrane to provide a retentate stream enriched in the protein or enzyme to recover the protein or enzyme.

13. A method of water purification comprising: passing a feedwater through the separation module of claim 1 such that the feedwater contacts a first side of the porous membrane with a pressure greater than osmotic pressure to produce purified water.

14. A water pretreatment system comprising the separation module of claim 1, designed for concentrating a feed and diluting a recirculating hypertonic solution to produce a slipstream; and a water makeup element for receiving the slipstream and combining the slipstream with the hypertonic solution to provide solutes to the recirculating hypertonic solution, herein the recirculating hypertonic solution is suitable for desalination.

15. A method of pretreating water, the method comprising:
receiving a feedwater; separating the feed water into a concentrator feed and a slipstream;
processing the concentrator feed in a concentrator comprising the separation module of claim 1 to generate a hypertonic solution; combining the slipstream and the hypertonic solution to generate an effluent capable of decomposition into purified water and a recirculating hypertonic solution.

16. A system for separating water-insoluble oil from oil-containing wastewater, the system comprising the separation module of claim 1.

17. (canceled)

18. An ultrafiltration device, the device comprising: a filter housing for a separation module, the filter housing comprising an inlet and an outlet, and a bundle of the tubular or capillary porous membranes of claim 5 fitted in the filter housing, the tubular or capillary membranes being permanently hydrophilic, whereby the tubular or capillary membranes are open at a first inlet end and sealed at the other end and are, at the first end, held in a membrane holder which closes off the space in between the tubular or capillary membranes and the filter housing wherein the pore size of the tubular or capillary membranes decreases in the direction of the liquid flow.

19. An apparatus for purification of a liquid by membrane distillation comprising the separation module of claim 1, wherein the separation module comprises a feed channel, a distillate channel, and a retentate channel, wherein the distillate channel and the retentate channel are separated by the porous membrane.

20. The apparatus for purification of a liquid by membrane distillation of claim 19, whereby the apparatus comprises a segment comprising a first distribution chamber for a feed liquid to be supplied, a second distribution chamber located opposite the first distribution chamber for feed liquid to be discharged, a third distribution chamber for retentate stream to be supplied and a fourth distribution chamber opposite the third the third distribution chamber for the retentate stream to be discharged, whereby the segment is provided with a first pump for pumping the feed stream pressure into the segment and a second pump which is arranged downstream the second distribution chamber for pumping the retentate stream under pressure into the retentate channel, the wall between the feed channel and the distillate channel comprises a condenser surface in the form of a non-porous membrane, and the wall between the retentate channel and the distillate channel comprises the porous membrane, and wherein inside the retentate channel a further channel is arranged for allowing a fluid stream to be brought into heat transfer contact with the retentate stream.

21. The porous membrane of claim 1, wherein the poly(phenylene ether) copolymer comprises:
80 to 20 mole percent repeat units derived from 2,6-dimethylphenol; and
20 to 80 mole percent repeat units derived from 2-methyl-6-phenylphenol, and
has an intrinsic viscosity of 0.7 to 1.5 deciliters per gram, when measured in chloroform at 25° C. and a weight average molecular weight of 100,000 to 500,000 daltons, as measured in chloroform by gel permeation chromatography against polystyrene standards.
